(19) United States
(12) Patent Application Publication
(10) Pub. No.: US 2015/0250837 A1
(43) Pub. Date: Sep. 10, 2015

(54) ONCOLYTIC VIRUS ENCODING PD-1 BINDING AGENTS AND USES OF THE SAME

(71) Applicant: MORNINGSIDE TECHNOLOGY VENTURES LTD., Monaco (MC)

(72) Inventors: Westley B. Nolin, Stoughton, MA (US); Shi Chung Ng, Indianapolis, IN (US); Jason R. Dinges, Boston, MA (US); Gerald L. Chan, Newton Centre, MA (US)

(21) Appl. No.: 14/429,673

(22) PCT Filed: Sep. 19, 2013
(86) PCT No.: PCT/US13/60716

§ 371 Date: Mar. 19, 2015

Related U.S. Application Data
(60) Provisional application No. 61/703,579, filed on Sep. 20, 2012.

(51) Int. Cl. A61K 35/768; A61K 35/763; A61K 35/761; C12N 7/00; C07K 16/28

(52) U.S. Cl. CPC A61K 35/768; C12N 7/00; C07K 16/2803; A61K 35/761; A61K 35/763; C12N 2710/24132; C12N 2710/10032; C12N 2710/16632; C12N 2710/10043; C12N 2710/16643; C12N 2710/24143; C07K 2317/76; C12N 2710/10071; C12N 2710/16671; C12N 2710/24171

(57) ABSTRACT

The present disclosure concerns the use of oncolytic viruses for the treatment of cancer. In particular, the use of a herpes simplex virus, a vaccinia virus, or an adenovirus containing a gene encoding a PD-1 binding agent, such as a scFv polypeptide, to achieve a particular degree of oncolysis is described. In some embodiments, the oncolytic virus expressing the PD-1 binding agent is effective at inducing immune responses that kill cancer cells at distant sites from the primary tumor. An oncolytic virus can also be engineered to be less toxic or damaging to non-cancer cells by mutation or modification of gene products such that the alterations render the viruses better able to infect the host, less toxic, and/or better able to selectively infect cancer cells.

### CROSS-REFERENCE TO RELATED APPLICATIONS

This application claims the benefit of and priority to U.S. Application No. 61/703,579, filed Sep. 20, 2012, which is incorporated herein by reference in its entirety.

### TECHNICAL FIELD

The present technology relates generally to the treatment of cancer using oncolytic viruses. In particular, the present technology relates to the preparation and use of recombinant viruses that carry genes for the expression of PD-1 binding agents.

### BACKGROUND

The following description is provided to assist the understanding of the reader. None of the information provided or references cited is admitted to be prior art to the present invention.

Cancer is diagnosed in more than 12 million people every year worldwide. In spite of numerous advances in medical research, cancer accounts for approximately 13% of all deaths. In industrialized nations, roughly one in five persons will die of cancer. Oncolytic virus therapy has emerged as a viable approach to specifically kill tumor cells. Unlike conventional gene therapy, it uses replication competent viruses that are able to spread through tumor tissue by virtue of viral replication and concomitant cell lysis. These viruses have been engineered to selectively replicate and kill cancer cells.

Malignant tumors are intrinsically resistant to conventional therapies and represent significant therapeutic challenges. For instance, micrometastases can become established at a very early stage in the development of primary tumors and seed distal tissue sites prior to clinical detection. Therefore, at the time of diagnosis many cancer patients already have microscopic metastasis. Tumor-reactive T cells can seek out and destroy these micrometastases and spare the surrounding healthy tissues. However, naturally existing T cell responses against malignancies are often not sufficient to cause regression of the primary or metastatic tumors. Using oncolytic viruses that express immunomodulatory proteins to break tolerance and to generate T cells capable of rejecting tumors may represent an approach to clearing metastatic tumor cells. Likewise, strategies designed to further enhance the potency of oncolytic viruses will increase their chance of clinical success.

### SUMMARY

The present disclosure concerns the use of oncolytic viruses for the treatment of cancer. In one aspect, the technology provides a recombinant oncolytic virus comprising a heterologous nucleic acid sequence encoding a PD-1 binding agent, wherein the heterologous nucleic acid sequence is stably incorporated into the genome of the oncolytic virus. In one embodiment, the PD-1 binding agent is an anti-PD-1 binding protein that antagonizes the activity of PD-1.

In one embodiment, the binding protein is selected from the group consisting of a natural ligand, a genetically modified ligand, a recombinant soluble domain of a natural receptor and a modified version thereof, a peptide ligand, a polypeptide ligand, an antibody molecule and fragments and derivatives thereof, and an antibody-like molecule. In one embodiment, the PD-1 binding agent is an anti-PD-1 single chain antibody.

In one embodiment, the oncolytic virus is selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus.

In one embodiment, the virus is HSV. In one embodiment, the HSV is selected from the group consisting of: HSV-1 or HSV-2. In one embodiment, the HSV genome has a mutation in each ICP34.5 locus such that the HSV cannot express a functional ICP34.5 gene product. In one embodiment, the HSV is HSV-2 and the HSV-2 genome encodes a modified ICP10 polypeptide having ribonucleotide reductase activity, but lacking protein kinase activity. In one embodiment, the modified ICP10 polypeptide has a deletion in the protein kinase domain of ICP10. In one embodiment, the HSV genome has an inactivating mutation in the ICP47 locus. In one embodiment, the HSV genome has an inactivating mutation in the ICP6 locus.

In one embodiment, the virus is a vaccinia virus. In one embodiment, the virus comprises an inactivating mutation in a thymidine kinase (TK) gene to produce a negative TK phenotype. In one embodiment, the vaccinia virus does not express functional vaccinia growth factor (VGF). In one embodiment, the vaccinia virus does not express functional B13R. In one embodiment, the vaccinia virus does not express functional B8R. In one embodiment, the vaccinia virus does not express functional A47. In one embodiment, the virus is an adenovirus.

In one embodiment, the virus further comprises a gene encoding an immunomodulatory protein selected from the group consisting of: tumor necrosis factor, interferon alpha, interferon gamma, IL-2, IL-12, IL-17 and GM-CSF.

In one embodiment, the heterologous nucleic acid sequence encoding the PD-1 binding agent is operably linked to a CMV promoter or a late stage viral promoter. In one embodiment, the heterologous nucleic acid sequence encoding the PD-1 agent is operably linked to an early/late promoter (P_{SEL}), a vaccinia 11 kDa protein promoter (p11), or a 7.5 kDa protein promoter (p7.5).

In another aspect, the technology provides an isolated recombinant herpes simplex virus 1 (HSV-1) comprising a heterologous nucleic acid sequence encoding a PD-1 binding agent, wherein the heterologous nucleic acid sequence is stably incorporated into the genome of the HSV-1 virus, and wherein the HSV-1 genome has a mutation in the ICP47 locus and a mutation in each ICP34.5 locus such that the HSV cannot express a functional ICP34.5 gene product. In one embodiment, the PD-1 binding agent is an anti-PD-1 single chain antibody. In one embodiment, the virus further comprises a gene encoding GM-CSF.

In another aspect, the technology provides an isolated recombinant herpes simplex virus 2 (HSV-2) comprising a heterologous nucleic acid sequence encoding a PD-1 binding agent, wherein the heterologous nucleic acid sequence is stably incorporated into the genome of the HSV-2 virus, wherein the HSV-2 genome encodes a modified ICP10 polypeptide having an inactivating mutation in the protein kinase domain of ICP10. In one embodiment, the modified ICP10 polypeptide lacks amino acids 106-445 of the native ICP10 polypeptide. In one embodiment, the HSV-2 genome has a mutation in the ICP47 locus. In one embodiment, the HSV-2 genome has a mutation in each ICP34.5 locus such that the HSV cannot express a functional ICP34.5 gene product. In a suitable embodiment, the HSV-2 virus is FusOn-H2. In one embodiment, the PD-1 binding agent is an anti-PD-1 single chain antibody. In one embodiment, the virus further comprises a gene encoding GM-CSF, IL-15 or IL-24.

In another aspect, the technology provides an isolated recombinant vaccinia virus comprising a gene encoding a heterologous nucleic acid sequence encoding a PD-1 binding agent, wherein the heterologous nucleic acid sequence is stably incorporated into the genome of the vaccinia virus. In one embodiment, the PD-1 binding agent is an anti-PD-1 single chain antibody.

In another aspect, the technology provides an isolated recombinant adenovirus comprising a gene encoding a heterologous nucleic acid sequence encoding a PD-1 binding agent, wherein the heterologous nucleic acid sequence is stably incorporated into the genome of the adenovirus. In one embodiment, the PD-1 binding agent is an anti-PD-1 single chain antibody. In one embodiment, the adenovirus is selected from the group comprising adenovirus serotypes: Ad1, Ad2, Ad3, Ad4, Ad5, Ad11, Ad35 and Ad41, or chimeric adenovirus serotypes.

In another aspect, the technology provides a pharmaceutical composition comprising an effective amount of the oncolytic virus described herein and a pharmaceutically acceptable carrier. In one embodiment, the composition is formulated for parenteral administration. In one embodiment, the composition is formulated for intratumoral administration.

In another aspect, the technology provides a method of treating cancer comprising administering to a subject in need thereof an effective amount of the oncolytic virus described herein.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is a graph showing the relative tumor volume over time of B16F10 melanoma mice receiving either placebo, VACV-TK⁻, J43, or VACV-TK⁻+J43.

FIG. 2 is a graph showing a Kaplan-Meier survival curve of B16F10 melanoma mice receiving either placebo, VACV-TK⁻, J43, or VACV-TK⁻+J43.

FIG. 3 is a graph showing the relative tumor volume over time of B16F10 melanoma mice receiving either placebo, vvDD, J43, or vvDD+J43.

FIG. 4 is a graph showing the relative tumor volume over time of B16F10 melanoma mice receiving either placebo, VACV-TK⁻, vvDD, or vvDD-mGMCSF.

FIG. 5 is a graph showing a Kaplan-Meier survival curve of B16F10 melanoma mice receiving either placebo, VACV-TK⁻, vvDD, J43, vvDD+J43, vvDD-mGMCSF.

FIG. 6 is a schematic diagram showing the various viral constructs used in the Examples.

FIG. 7A is a schematic of the pTK-J43 shuttle plasmid (encoding the J43 antibody). FIG. 7B is a graph showing the transient expression of J43, a mouse PD-1 antibody via transient transfection in HEK293 cells.

### DETAILED DESCRIPTION

It is to be appreciated that certain aspects, modes, embodiments, variations and features of the invention are described below in various levels of detail in order to provide a substantial understanding of the present technology. The practice of the present technology employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 7th ed., 2004 or 8th ed.). Further information on cancer may be found in The Biology of Cancer, Weinberg, R A, et al., Garland Science, 2006.

The present technology is described herein using several definitions, as set forth throughout the specification. Unless otherwise stated, the singular forms “a,” “an,” and “the” include the plural reference. For example, a reference to “a virus” includes a plurality of virus particles, and a reference to “a nucleic acid” is a reference to one or more nucleic acids.

As used herein, the term “administration” of an agent or drug to a subject includes any route of introducing or delivering to a subject a compound to perform its intended function. Administration can be carried out by any suitable route, including orally, intratumorally, intracranially, parenterally (intravenously, intramuscularly, intraperitoneally, or subcutaneously), rectally, or topically. Administration includes self-administration and the administration by another.

As used herein, the term “antibody” means a polypeptide comprising a framework region from an immunoglobulin gene or fragments thereof that specifically binds and recognizes an antigen, e.g., a PD-1 polypeptide. Use of the term antibody is meant to include whole antibodies, including single-chain antibodies, and antigen-binding fragments thereof. The term “antibody” includes bispecific antibodies and multispecific antibodies so long as they exhibit the desired biological activity or function.

As used herein, the term “cancer” refers to a class of diseases of humans (and animals) characterized by uncontrolled cellular growth. As used herein, “cancer” is used interchangeably with the terms “tumor,” “malignancy,” “hyperproliferation” and “neoplasm(s).” The term “cancer cell(s)” is interchangeable with the terms “tumor cell(s),” “malignant cell(s),” “hyperproliferative cell(s),” and “neoplastic cell(s)” unless otherwise explicitly indicated. Similarly, the terms “hyperproliferative,” “hyperplastic,” “malignant” and “neoplastic” are used interchangeably, and refer to those cells in an abnormal state or condition characterized by rapid proliferation. Collectively, these terms are meant to include all types of hyperproliferative growth, hyperplastic growth, neoplastic growth, cancerous growths or oncogenic processes, metastatic tissues or malignantly transformed cells, tissues, or organs, irrespective of histopathologic type or stage of invasiveness.

As used herein, the term “chemotherapy” refers to any therapy that includes natural or synthetic chemotherapeutic agents now known or to be developed in the medical arts. Examples of chemotherapeutic agents include the numerous cancer drugs that are currently available. However, chemotherapy also includes any drug, natural or synthetic, that is intended to treat a disease state. In certain embodiments, chemotherapy may include the administration of several state of the art drugs intended to treat the disease state. Examples include chemotherapy with doxorubicin, cisplatin, 5-fluorouracil, fludarabine and bendamustine.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Cabilly et al. U.S. Pat. No. 4,816,567; Cabilly et al., European Patent Application 125,023; Better et al., Science 240: 1041-1043, 1988; Liu et al., Proc Natl Acad Sci USA 84: 3439-3443, 1987; Liu et al., J Immunol 139: 3521-3526, 1987; Sun et al., Proc Natl Acad Sci USA 84: 214-218, 1987; Nishimura et al., Cancer Res 47: 999-1005, 1987; Wood et al., Nature 314: 446-449, 1885; and Shaw et al., J Natl Cancer Inst 80: 1553-1559, 1988.

As used herein, the term “effective amount” or “pharmaceutically effective amount” or “therapeutically effective amount” or “prophylactically effective amount” of a composition, is a quantity sufficient to achieve a desired therapeutic and/or prophylactic effect, e.g., an amount which results in the prevention of, or a decrease in, the symptoms associated with a disease that is being treated, e.g., a cancer. The amount of a composition administered to the subject will depend on the type and severity of the disease and on the characteristics of the individual, such as general health, age, sex, body weight and tolerance to drugs. It will also depend on the degree, severity and type of disease. In some embodiments, an effective amount of an oncolytic virus may be administered to a subject having cancer in an amount sufficient to exert oncolytic activity, causing attenuation or inhibition of tumor cell proliferation leading to primary and/or metastatic tumor regression.

As used herein, the term “humanized” refers to forms of non-human (e.g., murine) antibodies that are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, etc.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567).

As used herein, the term “PD-1” is an acronym for the Programmed Cell Death 1 protein, a 50-55 kDa type I transmembrane receptor originally identified by subtractive hybridization of a mouse T cell line undergoing apoptosis (Ishida et al., 1992, Embo J. 11:3887-95). PD-1 is expressed on activated T, B, and myeloid lineage cells (Greenwald et al., 2005, Annu. Rev. Immunol. 23:515-48; Sharpe et al., 2007, Nat. Immunol. 8:239-45). The amino acid sequence of human PD-1 is GenBank Accession No. NP_{—}005009.2. The amino acid sequence of murine PD-1 is GenBank Accession No. AAI19180.1.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “oncolytic virus” refers to a virus capable of selectively replicating in and slowing the growth or inducing the death of a cancerous or hyperproliferative cell, either in vitro or in vivo, while having no or minimal effect on normal cells. Exemplary oncolytic viruses include vesicular stomatitis virus (VSV), Newcastle disease virus (NDV), herpes simplex virus (HSV), reovirus, measles virus, retrovirus, influenza virus, Sinbis virus, vaccinia virus, adenovirus, or the like (see, e.g., Kirn et al., Nat. Med. 7:781 (2001); Coffey et al., Science 282:1332 (1998); Lorence et al., Cancer Res. 54:6017 (1994); and Peng et al., Blood 98:2002 (2001)).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. In a particular embodiment, the polynucleotide contains sequences encoding a PD-1 binding agent, such as an anti-PD-1 antibody (e.g., an anti-PD-1 scFv).

As used herein, the terms “polypeptide”, “peptide” and “protein” are used interchangeably to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature. In a particular embodiment, the polypeptide contains polypeptide sequences of a PD-1 binding agent, such as an anti-PD-1 antibody (e.g., an anti-PD-1 scFv).

As used herein, the term “recombinant” when used with reference, e.g., to a cell, virus, nucleic acid, protein, or vector, indicates that the cell, virus, nucleic acid, protein or vector, has been modified by the introduction of a heterologous nucleic acid or protein or the alteration of a native nucleic acid or protein, or that the material is derived from a cell so modified. Thus, e.g., recombinant viruses express genes that are not found within the native (non-recombinant) form of the virus or express native genes that are otherwise abnormally expressed, under expressed or not expressed at all. Thus, when referring to a “recombinant HSV” or “recombinant vaccinia” it is meant that the HSV or vaccinia has been genetically altered, e.g., by the addition or insertion of a selected gene, e.g., a PD-1 binding agent.

As used herein, the term “subject” refers to an organism administered one or more active agents. Typically, the subject is a mammal, such as an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like). Typically, the terms “subject” and “patient” are used interchangeably herein in reference to a human subject.

As used herein, the terms “treating” or “treatment” or “alleviation” refers to both therapeutic treatment and prophylactic or preventative measures, wherein the object is to prevent or slow down (lessen) the targeted pathologic condition or disorder. For example, a subject is successfully “treated” for a cancer, if after receiving a therapeutic amount of the oncolytic virus compositions described herein, the subject shows observable and/or measurable reduction in or absence of one or more signs and symptoms of the cancer, e.g., reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, of one or more of the symptoms associated with the specific cancer; reduced morbidity and mortality, and improvement in quality of life issues.

### Overview

Viruses have been shown to have utility in a variety of applications in biotechnology and medicine. An oncolytic virus is a virus that preferentially infects and lyses cancer cells, and has at least three functions in cancer therapy: (1) directly destroying the tumor cells by viral lysis, (2) serving as a vector for expressing heterologous proteins in the tumor site, and (3) the presentation of autologous tumor antigens to prime/activate the immune system. Oncolytic virus selectivity for cancer cells can occur either during infection or during replication. Tumor-selective viruses can be engineered by altering viral surface proteins that recognize specific cellular receptors, allowing the virus to specifically enter cancer cells. Replication selectivity can be accomplished by modifying the viral genes that are required for efficient replication, so that the virus can only replicate in cells that have disruptions in normal homeostatic pathways, such as tumor-suppressor defects or activation of oncogenic pathways.

In one aspect, the present technology relates to an improved method for eliciting an immune response in patients with tumors. A challenge in developing an effective and durable immunotherapy is to devise an approach to increase the number or enhance the function of tumor-specific T cells that may detect and destroy microscopic metastatic cells before they become clinically problematic. It is also important to elicit immune memory against the tumor. In some embodiments, the disclosure provides a method of eliciting an antitumor immune response in a patient presenting with, or at risk of developing, multiple metastatic tumors by administering an oncolytic virus (e.g., HSV, vaccinia virus, or adenovirus) that expresses a PD-1 binding agent, such as a single chain anti-PD-1 antibody, that antagonizes the activity of PD-1. In other embodiments, the oncolytic virus expresses an agent that antagonizes the binding of the PD-1 ligands to the receptor, e.g., anti-PD-L1 and/or PD-L2 antibodies, PD-L1 and/or PD-L2 decoys, or a soluble PD-1 receptor.

The PD-1 signaling pathway plays an important role in tumor-associated immune dysfunction. Infection and lysis of the tumor cells can invoke a highly specific antitumor immune response which kills cells of the inoculated tumor, as well as cells of distant, established, non-inoculated tumors. Tumors and their microenvironments have developed mechanisms to evade, suppress and inactivate the natural anti-tumor immune response. For example, tumors may down-regulate targeted receptors, encase themselves in a fibrous extracellular stromal matrix or up-regulate host receptors or ligands involved in the activation or recruitment of regulatory immune cells. Natural and/or adaptive T regulatory cells (Tregs) have been implicated in tumor-mediated immune suppression. Without wishing to be limited by theory, PD-1 blockade may inhibit Treg activity and improve the efficacy of tumor-reactive CTLs. Further aspects of the technology will be described in further detail below. PD-1 blockade may also stimulate the anti-tumor immune response by blocking the inactivation of T-cells (CTLs and helper) and B-cells.

In one aspect, the present technology provides an oncolytic virus that carries a gene encoding a PD-1 binding agent. Programmed Cell Death 1 (PD-1) is a 50-55 kDa type I transmembrane receptor originally identified by subtractive hybridization of a mouse T cell line undergoing apoptosis (Ishida et al., 1992, Embo J. 11:3887-95). A member of the CD28 gene family, PD-1 is expressed on activated T, B, and myeloid lineage cells (Greenwald et al., 2005, Annu. Rev. Immunol. 23:515-48; Sharpe et al., 2007, Nat. Immunol. 8:239-45). Recent publications also suggest that PD-1 is also expressed by subsets of DCs, exhausted T-cells and CD4+ T-Regs. FASEB J. 2008 October; 22(10):3500-8 and Immunol Rev. 2010 July; 236:219-42. Human and murine PD-1 share about 60% amino acid identity with conservation of four potential N-glycosylation sites and residues that define the Ig-V domain. Two ligands for PD-1 have been identified, PD ligand 1 (PD-L1) and ligand 2 (PD-L2); both belong to the B7 superfamily. PD-L1 is expressed on many cell types, including T, B, endothelial and epithelial cells, and antigen presenting cells. In contrast, PD-L2 is narrowly expressed on professional antigen presenting cells, such as dendritic cells and macrophages.

PD-1 negatively modulates T cell activation, and this inhibitory function is linked to an immunoreceptor tyrosine-based inhibitory motif (ITIM) of its cytoplasmic domain (Parry et al., 2005, Mol. Cell. Biol. 25:9543-53). Disruption of this inhibitory function of PD-1 can lead to autoimmunity. The reverse scenario can also be deleterious. Sustained negative signals by PD-1 have been implicated in T cell dysfunctions in many pathologic situations, such as tumor immune evasion and chronic viral infections.

Host anti-tumor immunity is mainly affected by tumor-infiltrating lymphocytes (TILs) (Galore et al., 2006, Science 313:1960-4). Multiple lines of evidence have indicated that TILs are subject to PD-1 inhibitory regulation. First, PD-L1 expression is confirmed in many human and mouse tumor lines and the expression can be further upregulated by IFN-γ in vitro (Dong et al., 2002, Nat. Med. 8:793-800). Second, expression of PD-L1 by tumor cells has been directly associated with their resistance to lysis by anti-tumor T cells in vitro (Blank et al., 2004, Cancer Res. 64:1140-5). Third, PD-1 knockout mice are resistant to tumor challenge (Iwai et al., 2005, Int. Immunol. 17:133-44) and T cells from PD-1 knockout mice are highly effective in tumor rejection when adoptively transferred to tumor-bearing mice (Blank et al., supra). Fourth, blocking PD-1 inhibitory signals by a monoclonal antibody can potentiate host anti-tumor immunity in mice (Iwai et al., supra; Hirano et al., 2005, Cancer Res. 65:1089-96). Fifth, high degrees of PD-L1 expression in tumors (detected by immunohistochemical staining) are associated with poor prognosis for many human cancer types (Hamanishi et al., 2007, Proc. Natl. Acad. Sci. USA 104:3360-5).

Oncolytic virotherapy is an effective method to shape the host immune system by expanding T or B cell populations specific for tumor-specific antigens that are released following oncolysis. The immunogenicity of the tumor-specific antigens is largely dependent on the affinity of host immune receptors (B-cell receptors or T-cell receptors) to antigenic epitopes and the host tolerance threshold. High affinity interactions will drive host immune cells through multiple rounds of proliferation and differentiation to become long-lasting memory cells. The host tolerance mechanisms will counterbalance such proliferation and expansion in order to minimize potential tissue damage resulting from local immune activation. PD-1 inhibitory signals are part of such host tolerance mechanisms, supported by following lines of evidence. First, PD-1 expression is elevated in actively proliferating T cells, especially those with terminal differentiated phenotypes, i.e., effector phenotypes. Effector cells are often associated with potent cytotoxic function and cytokine production. Second, PD-L1 is important to maintain peripheral tolerance and to limit overly active T cells locally. Therefore, PD-1 inhibition using a PD-1 binding agent expressed in the tumor microenvironment can be an effective strategy to increase the activity of TIL and stimulate an effective and durable anti-tumor immune response.

### PD-1 Binding Agents

In one aspect, the present technology provides an oncolytic virus comprising a heterologous nucleic acid encoding a PD-1 binding agent. In some embodiments, the PD-1 binding agents contain an antibody variable region providing for specific binding to a PD-1 epitope. The antibody variable region can be present in, for example, a complete antibody, an antibody fragment, and a recombinant derivative of an antibody or antibody fragment. The term “antibody” describes an immunoglobulin, whether natural or partly or wholly synthetically produced. Thus, PD-1 binding agents of the present technology include any polypeptide or protein having a binding domain which is specific for binding to a PD-1 epitope.

Different classes of antibodies have different structures. Different antibody regions can be illustrated by reference to IgG. An IgG molecule contains four polypeptide chains, two longer length heavy chains and two shorter light chains that are inter-connected by disulfide bonds. The heavy and light chains each contain a constant region and a variable region. A heavy chain is comprised of a heavy chain variable region (V_{H}) and a heavy chain constant region (CH₁, CH₂ and CH₃). A light chain is comprised of a light chain variable region (V_{L}) and a light chain constant region (C_{L}). There are three hypervariable regions within the variable regions that are responsible for antigen specificity. (See, for example, Breitling et al., Recombinant Antibodies, John Wiley & Sons, Inc. and Spektrum Akademischer Verlag, 1999; and Lewin, Genes IV, Oxford University Press and Cell Press, 1990.)

The hypervariable regions are generally referred to as complementarity determining regions (“CDR”) and are interposed between more conserved flanking regions referred to as framework regions (“FW”). There are four (4) FW regions and three (3) CDRs that are arranged from the NH₂ terminus to the COOH terminus as follows: FW1, CDR1, FW2, CDR2, FW3, CDR3, FW4. Amino acids associated with framework regions and CDRs can be numbered and aligned by approaches described by Kabat et al., Sequences of Proteins of Immunological Interest, U.S. Department of Health and Human Services, 1991; C. Chothia and A. M. Lesk, J Mol Biol 196(4):901 (1987); or B. Al-Lazikani, et al., J Mol Biol 273(4): 27, 1997. For example, the framework regions and CDRs can be identified from consideration of both the Kabat and Chothia definitions. The variable regions of the heavy and light chains contain a binding domain that interacts with an antigen. The two heavy chain carboxyl regions are constant regions joined by disulfide bonding to produce an Fc region. The Fc region is important for providing effector functions. (Presta, Advanced Drug Delivery Reviews 58:640-656, 2006.) Each of the two heavy chains making up the Fc region extends into different Fab regions through a hinge region.

PD-1 binding agents typically contain an antibody variable region. Such antibody fragments include but are not limited to (i) a Fab fragment, a monovalent fragment consisting of the V_{H}, V_{L}, C_{H} and C_{L} domains; (ii) a Fab₂ fragment, a bivalent fragment comprising two Fab fragments linked by a disulfide bridge at the hinge region; (iii) a Fd fragment consisting of the V_{H}, and C_{H1} domains; (iv) a Fv fragment consisting of the V_{H} and V_{L} domains of a single arm of an antibody; (v) a dAb fragment, which comprises either a V_{H} or V_{L} domain; (vi) a scAb, an antibody fragment containing V_{H} and V_{L} as well as either C₁ or C_{H1}; and, (vii) artificial antibodies based upon protein scaffolds, including but not limited to fibronectin type III polypeptide antibodies (e.g., see U.S. Pat. No. 6,703,199). Furthermore, although the two domains of the Fv fragment, V_{L} and V_{H}, are coded for by separate genes, they can be joined using recombinant methods by a synthetic linker that enables them to be made as a single protein chain in which the V_{L} and V_{H} regions pair to form monovalent molecules, known as single chain Fv (scFv). Thus, the antibody variable region can be present in a recombinant derivative. Examples of recombinant derivatives include single-chain antibodies, diabody, triabody, tetrabody, and miniantibody. A PD-1 binding agent can also contain one or more variable regions recognizing the same or different epitopes.

In some embodiments, PD-1 binding agents are encoded by an oncolytic virus produced using recombinant nucleic acid techniques. Different PD-1 binding agents can be produced by different techniques, including, for example, a single chain protein containing a V_{H} region and V_{L} region connected by a linker sequence, such as a scFv, and antibodies or fragments thereof; and a multi-chain protein containing a V_{H} and V_{L} region on separate polypeptides. Recombinant nucleic acid techniques involve constructing a nucleic acid template for protein synthesis. Suitable recombinant nucleic acid techniques are well known in the art. (See, for example, Ausubel, Current Protocols in Molecular Biology, John Wiley, 2005; Harlow et al., Antibodies, A Laboratory Manual, Cold Spring Harbor Laboratory, 1988.) Recombinant nucleic acid encoding a PD-1 binding protein can be expressed in a cell that has been infected with an oncolytic virus and released into the tumor microenvironment upon viral lysis. The cell in effect serves as a factory for the encoded protein.

A nucleic acid comprising one or more recombinant genes encoding for either or both of a PD-1 binding agent V_{H}, region or V_{L} region can be used to produce a complete binding protein binding to PD-1. A complete binding protein can be provided, for example, using a single gene to encode a single chain protein containing a V_{H} region and V_{L} region connected by a linker, such as a scFv, or using multiple recombinant regions to, for example, produce both V_{H} and V_{L} regions.

A recombinant gene encoding the PD-1 binding agent contains nucleic acid encoding a protein along with regulatory elements for protein expression. Generally, the regulatory elements that are present in a recombinant gene include a transcriptional promoter, a ribosome binding site, and a terminator. A promoter is defined as a DNA sequence that directs RNA polymerase to bind to DNA and initiate RNA synthesis. A strong promoter is one which causes mRNAs to be initiated at high frequency. A suitable element for processing in eukaryotic cells is a polyadenylation signal. Antibody associated introns may also be present. Examples of expression cassettes for antibody or antibody fragment production are well known in art. (E.g., Persic et al., 1997, Gene 187:9-18; Boel et al., 2000, J Immunol. Methods 239:153-166; Liang et al., 2001, J. Immunol. Methods 247:119-130; Tsurushita et al., 2005, Methods 36:69-83.)

Appropriate regulatory elements can be selected by those of ordinary skill in the art based on, for example, the desired tissue-specificity and level of expression. For example, a cell-type specific or tumor-specific promoter can be used to limit expression of a gene product to a specific cell type. In addition to using tissue-specific promoters, local administration of the viruses can result in localized expression and effect. Examples of non-tissue specific promoters that can be used include the early Cytomegalovirus (CMV) promoter (U.S. Pat. No. 4,168,062) and the Rous Sarcoma Virus promoter. Also, HSV promoters, such as HSV-1 IE and IE 4/5 promoters, can be used. In some embodiments, the promoter is selected from a promoter in Table 1 below.

TABLE 1
Exemplary Promoters

| Promoter | Tumor or Tissue Target |
|---|---|
| B-myb | Glioma liver metastasis |
| Nestin | Glioma |
| CEA (Carinoembryonic antigen) | Colon cancer |
| Albumin | Hepatoma |
| DF3/MUC1 (Mucin 1) | Pancreatic Cancer |
| Caponin | Leiomyosarcoma |

Examples of tissue-specific promoters that can be used in the technology include, for example, the prostate-specific antigen (PSA) promoter, which is specific for cells of the prostate; the desmin promoter, which is specific for muscle cells; the enolase promoter, which is specific for neurons; the beta-globin promoter, which is specific for erythroid cells; the tau-globin promoter, which is also specific for erythroid cells; the growth hormone promoter, which is specific for pituitary cells; the insulin promoter, which is specific for pancreatic beta cells; the glial fibrillary acidic protein promoter, which is specific for astrocytes; the tyrosine hydroxylase promoter, which is specific for catecholaminergic neurons; the amyloid precursor protein promoter, which is specific for neurons; the dopamine beta-hydroxylase promoter, which is specific for noradrenergic and adrenergic neurons; the tryptophan hydroxylase promoter, which is specific for serotonin/pineal gland cells; the choline acetyltransferase promoter, which is specific for cholinergic neurons; the aromatic L-amino acid decarboxylase (AADC) promoter, which is specific for catecholaminergic/5-HT/D-type cells; the proenkephalin promoter, which is specific for neuronal/spermatogenic epididymal cells; the reg (pancreatic stone protein) promoter, which is specific for colon and rectal tumors, and pancreas and kidney cells; and the parathyroid hormone-related peptide (PTHrP) promoter, which is specific for liver and cecum tumors, and neurilemoma, kidney, pancreas, and adrenal cells.

Examples of promoters that function specifically in tumor cells include the stromelysin 3 promoter, which is specific for breast cancer cells; the surfactant protein A promoter, which is specific for non-small cell lung cancer cells; the secretory leukoprotease inhibitor (SLPI) promoter, which is specific for SLPI-expressing carcinomas; the tyrosinase promoter, which is specific for melanoma cells; the stress inducible grp78/BiP promoter, which is specific for fibrosarcoma/tumorigenic cells; the AP2 adipose enhancer, which is specific for adipocytes; the a-1 antitrypsin transthyretin promoter, which is specific for hepatocytes; the interleukin-10 promoter, which is specific for glioblastoma multiform cells; the c-erbB-2 promoter, which is specific for pancreatic, breast, gastric, ovarian, and non-small cell lung cells; the a-B-crystallin/heat shock protein 27 promoter, which is specific for brain tumor cells; the basic fibroblast growth factor promoter, which is specific for glioma and meningioma cells; the epidermal growth factor receptor promoter, which is specific for squamous cell carcinoma, glioma, and breast tumor cells; the mucin-like glycoprotein (DF3, MUC1) promoter, which is specific for breast carcinoma cells; the mtsl promoter, which is specific for metastatic tumors; the NSE promoter, which is specific for small-cell lung cancer cells; the somatostatin receptor promoter, which is specific for small cell lung cancer cells; the c-erbB-3 and c-erbB-2 promoters, which are specific for breast cancer cells; the c-erbB4 promoter, which is specific for breast and gastric cancer; the thyroglobulin promoter, which is specific for thyroid carcinoma cells; the a-fetoprotein (AFP) promoter, which is specific for hepatoma cells; the villin promoter, which is specific for gastric cancer cells; and the albumin promoter, which is specific for hepatoma cells. In another embodiment, the TERT promoter or survivin promoter are used.

### Methods of Preparing a PD-1 Binding Agents

Techniques for generating antibodies directed to target polypeptides (e.g., PD-1) are well known to those skilled in the art. Examples of such techniques include, e.g., but are not limited to, those involving display libraries, xeno or humab mice, hybridomas, and the like. It should be understood that not only are naturally-occurring antibodies suitable as binding agents for use in accordance with the present disclosure, but recombinantly engineered antibodies and antibody fragments which are directed to PD-1 are also suitable.

PD-1 binding agents that can be subjected to the techniques set forth herein include monoclonal and polyclonal antibodies, and antibody fragments such as Fab, Fab′, F(ab′)₂, Fd, scFv, diabodies, antibody light chains, antibody heavy chains and/or antibody fragments. Generally, an antibody is obtained from an originating species. More particularly, the nucleic acid or amino acid sequence of the variable portion of the light chain, heavy chain or both, of an originating species antibody having specificity for PD-1 is obtained. Originating species is any species which was useful to generate the antibody or library of antibodies, e.g., rat, mice, rabbit, chicken, monkey, human, and the like.

PD-1 binding agents useful in the present technology include “human antibodies,” (e.g., antibodies isolated from a human) or “human sequence antibodies.” Human antibodies can be made by a variety of methods known in the art including phage display methods. Methods useful for the identification of nucleic acid sequences encoding members of multimeric polypeptide complex by screening polyphage particles have been described. Rudert et al., U.S. Pat. No. 6,667,150. Also, recombinant immunoglobulins can be produced. Cabilly, U.S. Pat. No. 4,816,567; Cabilly et al., U.S. Pat. No. 6,331,415 and Queen et al., Proc. Nat'l Acad. Sci. USA 86: 10029-10033, 1989. Techniques for generating and cloning monoclonal antibodies are well known to those skilled in the art.

Preparation of Polyclonal Antisera and Immunogens.

Methods of generating antibodies or antibody fragments typically include immunizing a subject (generally a non-human subject such as a mouse or rabbit) with the purified PD-1 polypeptide or with a cell expressing the PD-1 polypeptide. Any immunogenic portion of the PD-1 polypeptide can be employed as the immunogen. An appropriate immunogenic preparation can contain, e.g., a recombinantly-expressed PD-1 polypeptide or a chemically-synthesized PD-1 polypeptide. An isolated PD-1 polypeptide, or a portion or fragment thereof, can be used as an immunogen to generate a PD-1 antibody that binds to the PD-1 polypeptide, or a portion or fragment using standard techniques for polyclonal and monoclonal antibody preparation. The full-length PD-1 polypeptide can be used or, alternatively, the technology provides for the use of the PD-1 polypeptide fragments as immunogens. The PD-1 polypeptide comprises at least four contiguous amino acid residues of the amino acid sequence shown in GenBank Accession No. NP_{—}005009.2, and encompasses an epitope of the PD-1 polypeptide such that an antibody raised against the peptide forms a specific immune complex with the PD-1 polypeptide. Suitably, the antigenic peptide comprises at least 5, 8, 10, 15, 20, or 30 contiguous amino acid residues. Longer antigenic peptides are sometimes preferable over shorter antigenic peptides, depending on use and according to methods well known to those skilled in the art. Typically, the immunogen will be at least about 8 amino acyl residues in length, and preferably at least about 10 amino acid residues in length. Multimers of a given epitope are sometimes more effective than a monomer.

If needed, the immunogenicity of the PD-1 polypeptide (or fragment thereof) can be increased by fusion or conjugation to a hapten such as keyhole limpet hemocyanin (KLH) or ovalbumin (OVA). Many such haptens are known in the art. One can also combine the PD-1 polypeptide with a conventional adjuvant such as Freund's complete or incomplete adjuvant to increase the subject's immune reaction to the polypeptide. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. These techniques are standard in the art.

The subject is initially immunized with the PD-1 polypeptide to generate a primary immune response. A primary immune response, which is also described as a “protective” immune response, refers to an immune response produced in a subject as a result of some initial exposure (e.g., the initial “immunization”) to a particular antigen, e.g., a PD-1 polypeptide. A primary immune response can become weakened or attenuated over time and can even disappear or at least become so attenuated that it cannot be detected. Thus, a secondary or immune response can be elicited, e.g., to enhance an existing immune response that has become weakened or attenuated, or to recreate a previous immune response that has either disappeared or can no longer be detected. As an example, and not by way of limitation, a secondary immune response can be elicited by re-introducing to the subject an antigen, e.g., a PD-1 polypeptide, that elicited the primary immune response (e.g., by re-administrating a vaccine). The secondary or memory immune response can be either a humoral (antibody) response or a cellular response. A secondary or memory humoral response occurs upon stimulation of memory B cells that were generated at the first presentation of the antigen.

Following appropriate immunization, the PD-1 antibody, e.g., anti-PD-1 polyclonal antibody can be prepared from the subject's serum. If desired, the antibody molecules directed against the PD-1 polypeptide can be isolated from the mammal (e.g., from the blood) and further purified by well known techniques, such as Protein A chromatography to obtain the IgG fraction.

Monoclonal Antibody.

In one embodiment, the PD-1 binding agent is an anti-PD-1 monoclonal antibody. In some embodiments, the anti-PD-1 monoclonal antibody is a human, humanized, or chimeric anti-PD-1 monoclonal antibody. For preparation of monoclonal antibodies directed towards a particular PD-1 polypeptide, or derivatives, fragments, analogs or homologs thereof, any technique that provides for the production of antibody molecules by continuous cell line culture can be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: Monoclonal Antibodies and Cancer Therapy, Alan R. Liss, Inc., pp. 77-96). Human monoclonal antibodies can be utilized in the practice of the technology and can be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: Monoclonal Antibodies and Cancer Therap, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the PD-1 polypeptide. Alternatively, hybridomas expressing anti-PD-1 monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., PD-1 binding, can be used as expressed by the hybridoma, or a cDNA encoding it can be isolated, sequenced and manipulated in various ways.

Hybridoma Technique.

In one embodiment, the PD-1 binding agent of the technology is an anti-PD-1 monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981). Other methods for producing hybridomas and monoclonal antibodies are well known to those of skill in the art.

Expression of Recombinant PD-1 Antibody.

As noted above, the antibodies of the present technology can be produced through the application of recombinant DNA technology. Recombinant polynucleotide constructs encoding a PD-1 antibody of the present technology typically include an expression control sequence operably-linked to the coding sequences of anti-PD-1 antibody chains, including naturally-associated or heterologous promoter regions. As such, another aspect of the technology includes vectors containing one or more nucleic acid sequences encoding a PD-1 antibody. For recombinant expression of one or more the polypeptides, the nucleic acid containing all or a portion of the nucleotide sequence encoding the PD-1 antibody is inserted into an appropriate cloning vector, or an expression vector (i.e., a vector that contains the necessary elements for the transcription and translation of the inserted polypeptide coding sequence) by recombinant DNA techniques well known in the art and as detailed below.

In general, expression vectors useful in recombinant DNA techniques are often in the form of plasmids. In the present specification, “plasmid” and “vector” can be used interchangeably as the plasmid is the most commonly used form of vector. However, the technology is intended to include such other forms of expression vectors that are not technically plasmids, such as viral vectors (e.g., HSV, vaccina virus, replication defective retroviruses, adenoviruses and adeno-associated viruses), which serve equivalent functions. Such viral vectors permit infection of a subject and expression in that subject of a compound. Suitably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the PD-1 antibody, and the collection and purification of the PD-1 antibody, e.g., cross-reacting anti-PD-1 antibodies. Vectors can also encode signal peptide, e.g., pectate lyase, useful to direct the secretion of extracellular antibody fragments. See U.S. Pat. No. 5,576,195.

The recombinant expression vectors of the technology comprise a nucleic acid encoding a compound with PD-1 binding properties in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc.

Single Chain Antibodies.

In one embodiment, the antibody of the technology is a single chain anti-PD-1 antibody. Techniques can be adapted for the production of single-chain antibodies specific to a PD-1 polypeptide (see, e.g., U.S. Pat. No. 4,946,778). Examples of techniques which can be used to produce single-chain Fvs and antibodies include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies.

In one embodiment, the antibody is a chimeric anti-PD-1 antibody. In one embodiment, the antibody is a humanized anti-PD-1 antibody. In one embodiment, the donor and acceptor antibodies are monoclonal antibodies from different species. For example, the acceptor antibody is a human antibody (to minimize its antigenicity in a human), in which case the resulting CDR-grafted antibody is termed a “humanized” antibody. Recombinant anti-PD-1 antibodies, such as chimeric and humanized monoclonal antibodies, comprising both human and non-human portions, can be made using standard recombinant DNA techniques, and are within the scope of the technology. For some uses, including in vivo use of the antibody in humans, it is preferable to use chimeric, humanized, or human anti-PD-1 antibodies. Such chimeric and humanized monoclonal antibodies can be produced by recombinant DNA techniques known in the art. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine anti-PD-1 monoclonal antibody is digested with a restriction enzyme selected specifically to remove the sequence encoding the Fc constant region, and the equivalent portion of a cDNA encoding a human Fc constant region is substituted.

### Herpes Viruses

In one embodiment, the oncolytic virus comprising a gene encoding a PD-1 binding agent is a herpes virus. The oncolytic herpes viruses may be derived from several different types of herpes viruses. The Herpesviridae are a large family of DNA viruses that cause diseases in humans and animals. Herpes viruses all share a common structure and are composed of relatively large double-stranded, linear DNA genomes encoding 100-200 genes encased within an icosahedral protein cage called the capsid which is itself wrapped in a lipid bilayer membrane called the envelope. This particle is known as the virion. The large genome provides many non-essential sites for introducing one or more transgenes without inactivating the virus (e.g., without completely inhibiting infection or replication). However, it should be appreciated that viral vectors are suitably modified (e.g., replication conditional, attenuated) so that they do not have undesirable effects (e.g., kill normal cells, causes disease).

As used herein, “oncolytic herpes virus” refers to any one of a number of therapeutic viruses having a herpes virus origin that are useful for killing cancer cells, and/or inhibiting the growth of a tumor. Typically, an oncolytic herpes virus is a mutant version of a wild-type herpes virus, such as a replication-conditional herpes virus. Replication-conditional herpes viruses are designed to preferentially replicate in actively dividing cells, such as cancer cells. Thus, these replication-conditional viruses target cancer cells for oncolysis, and replicate in these cells so that the virus can spread to other cancer cells.

The herpes virus may comprise any one of a number of mutations that affect expression of a viral gene. In most cases, a mutation is in a virulence gene that contributes to the pathogenicity of the virus to a host organism. The mutation may be a point mutation, a deletion, an inversion, a substitution or an insertion. Typically, the mutation is an inactivating mutation, which refers to a mutation or alteration to a gene wherein the expression of that gene is significantly decreased, or wherein the gene product is rendered nonfunctional, or its ability to function is significantly decreased.

Several types of replication-conditional herpes virus mutants have been developed and are useful in aspects of the methods disclosed herein. For example, one aspect involves viral mutants with defects in the function of a viral gene needed for nucleic acid metabolism, such as thymidine kinase (Martuza, R. L., et al., Science 252:854-856 (1991)), ribonucleotide reductase (RR) (Goldstein, D. J. & Weller, S. K., J. Virol. 62:196-205 (1988); Boviatsis, E. J., et al., Gene Ther. 1:323-331 (1994); Boviatsis, E. J., et al., Cancer Res. 54:5745-5751 (1994); Mineta, T., et al., Cancer Res. 54:3363-3366 (1994)), or uracil-N-glycosylase (Pyles, R. B. and Thompson, R. I., J. Virol. 68:4963-4972 (1994)). Another aspect involves viral mutants with defects in the function of the γ34.5 (ICP34.5) gene (Chambers, R., et al., Proc. Natl. Acad. Sci. USA 92:1411-1415 (1995)), which functions as a virulence factor by markedly enhancing the viral burst size of infected cells through suppression of the shutoff of host protein synthesis (Chou, J., et al., Science 250:1262-1266 (1990); Chou, J. and Roizman, B., Proc. Natl. Acad. Sci. USA 89:3266-3270 (1992)). Other examples include G207 (Mineta, T., et al., Nat. Med 1:938-943 (1995); U.S. Pat. No. 5,585,096, issued Dec. 17, 1996 to Martuza et al.), and MGH1 (Kramm, C. M., et al., Hum. Gene Ther. 8:2057-2068 (1997), which possess deletions of both copies of γ34.5 and an insertional mutation of ribonucleotide reductase.

In some embodiments, the oncolytic virus is a herpes simplex virus (HSV viruses), for example, HSV-1 (e.g., HSV-1 strain F or strain Patton) or HSV-2, that include an inactivating mutation in a virulence gene. In the case of herpes simplex viruses, this mutation can be an inactivating mutation in the γ34.5 gene, which is the major HSV neurovirulence determinant. In some embodiments, the oncolytic herpes virus includes an inactivating mutation in one or both copies of the γ34.5 genes. An additional HSV-based virus includes, in addition to an inactivating mutation in the γ34.5 locus, a second modification that results in down regulation of ICP47 expression. In another embodiment, the virus can include a mutation in the ICP6 gene, which encodes the large subunit of ribonucleotide reductase.

In some embodiments, the viruses can also include, optionally, sequences encoding a heterologous gene product, such as a PD-1 binding agent and/or another immunomodulatory protein. For example, a heterologous nucleic acid sequence can be inserted into a virus in a location that renders it under the control of a regulatory sequence of the virus.

In one embodiment, the oncolytic virus is a HSV-2 that contains an inactivating mutation in the protein kinase domain of the ICP10 gene. Thus, the modified ICP10 polynucleotide encodes for a polypeptide that has ribonucleotide reductase activity, but lacks protein kinase (PK) activity. Deletion of the PK domain from the ribonucleotide reductase gene severely compromises the ability of the virus to replicate in cells where there is no preexisting activated Ras signaling pathway (Smith et al (1998) J. Virol. 72(11):9131-9141). The ICP10 polynucleotide may be modified either by deleting at least some of the sequence required for encoding a functional PK domain, or replacing at least part of the sequence encoding the PK domain with a second polynucleotide. The region of the PK domain that is deleted/replaced may be any suitable region so long as the polypeptide encoded by the modified ICP10 polynucleotide retains ribonucleotide reductase activity and lacks protein kinase activity. In certain embodiments, though, the modification to the PK domain affects one or more of the eight PK catalytic motifs (amino acid residues 106-445 or 1-445), and/or the transmembrane (TM) region, and/or the invariant Lys (Lys176). An exemplary wild-type ICP10 polypeptide sequence is described as GenBank Accession No. 1813262A. In an exemplary embodiment, the HSV-2 virus is FusON-H2 (see WO 2007/002373).

One of skill in the art will recognize that any suitable method can be used for generating inactivating mutations in a gene of interest, including mutagenesis, polymerase chain reaction, homologous recombination, or any other genetic engineering technique known to a person of skill in the art. Mutation can involve modification of a nucleotide sequence, a single gene, or blocks of genes. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

In other embodiments, the gene of interest is modified using genetic recombination techniques to delete or replace at least part of the native sequence. For example, a second functional polynucleotide may be used to replace part or all of the gene of interest. This second functional polynucleotide may encode a PD-1 binding agent or another therapeutic agent. Exemplary, non-limiting examples of polynucleotides encoding for other therapeutic agents include tumor necrosis factor; interferon, alpha, beta, gamma; interleukin-2 (IL-2), IL-12, IL-15, IL-24, granulocyte macrophage-colony stimulating factor (GM-CSF), F42K, MIP-I, MIP-Iβ, MCP-I, RANTES, Herpes Simplex Virus-thymidine kinase (HSV-tk), cytosine deaminase, and caspase-3. In other embodiments, the HSV genome is modified by insertion of a polynucleotide encoding a reporter protein. Exemplary non-limiting polynucleotides encoding for reporter proteins include green fluorescent protein, enhanced green fluorescent protein, β-galactosidase, luciferase, and HSV-tk.

### Vaccinia Virus

In one embodiment, the oncolytic virus comprising a gene encoding a PD-1 binding agent is a vaccinia virus. Vaccinia virus is a large, complex enveloped virus having a linear double-stranded DNA genome of about 190 kilobases and encodes approximately 250 genes. Vaccinia is well-known for its role as a vaccine that eradicated smallpox. Vaccinia virus is unique among DNA viruses as it replicates only in the cytoplasm of the host cell. Therefore, the large genome is required to code for various enzymes and proteins needed for viral DNA replication. During replication, vaccinia produces several infectious forms which differ in their outer membranes: the intracellular mature virion (IMV), the intracellular enveloped virion (IEV), the cell-associated enveloped virion (CEV) and the extracellular enveloped virion (EEV). IMV is the most abundant infectious form and is thought to be responsible for spread between hosts. On the other hand, the CEV is believed to play a role in cell-to-cell spread and the EEV is thought to be important for long range dissemination within the host organism. A number of genes in vaccinia can be modified in order to improve its properties as an oncolytic virus, and are described in further detail below.

In one embodiment, one or more interferon-modulating genes are altered in the oncolytic vaccinia virus. For example, vaccinia virus encodes the secreted proteins B8R and B18R which bind interferon-γ and -α/β, respectively. An additional example of a vaccinia gene product that reduces interferon induction is the caspase-1 inhibitor B13R which inhibits activation of the interferon-γ-inducing factor IL-18. Accordingly, in one embodiment, the vaccinia virus has a mutation in an interferon-modulating gene selected from the group consisting of: B13R, B18R, B8R, vC12L, A53R, and E3L. In a suitable embodiment, the vaccinia virus has a mutation in the B13R gene.

In one embodiment, one or more complement control genes are altered in the oncolytic vaccinia virus. Poxviruses such as vaccinia have evolved to express gene products that are able to counteract the complement-mediated clearance of virus and/or virus-infected cells. These genes thereby prevent apoptosis and inhibit viral clearance by complement-dependent mechanisms, thus allowing the viral infection to proceed and viral virulence to be increased. For example, vaccinia virus complement control proteins (VCP; e.g., C21L) have roles in the prevention of complement-mediated cell killing and/or virus inactivation. VCP also has anti-inflammatory effects since its expression decreases leukocyte infiltration into virally-infected tissues. Accordingly, in one embodiment, the vaccinia virus has a mutation in a complement control polypeptides including, but are not limited to C3L or C21L.

In one embodiment, one or more TNF-modulating polypeptides are altered in the oncolytic vaccinia virus. Various strains of poxviruses, including some vaccinia virus strains, have evolved to express gene products that are able to counteract the TNF-mediated clearance of virus and/or virus-infected cells. The proteins encoded by these genes circumvent the proinflammatory and apoptosis inducing activities of TNF by binding and sequestering extracellular TNF, resulting in the inhibition of viral clearance. Because viruses are not cleared, the viral infection is allowed to proceed, and thus, viral virulence is increased. Various members of the poxvirus family express secreted viral TNF receptors (vTNFR). For example, several poxviruses encode vTNFRs, such as myxoma (T2 protein), cowpox and vaccinia virus strains, such as Lister, may encode one or more of the CrmB, CrmC (A53R), CrmD, CrmE, B28R proteins and/or equivalents thereof. Accordingly, in one embodiment, the vaccinia virus has a mutation in a TNF modulatory polypeptide including, but not limited to, A53R, B28R, and other polypeptides with similar activities or properties.

In one embodiment, one or more serine protease inhibitor polypeptides are altered in the oncolytic vaccinia virus. A major mechanism for the clearance of viral pathogens is the induction of apoptosis in infected cells within the host. As the infected cell dies, it is unable to continue to produce infectious virus. In addition, during apoptosis intracellular enzymes are released which degrade DNA. These enzymes can lead to viral DNA degradation and virus inactivation. Serpins (serine protease inhibitors) have roles in the prevention of various forms of apoptosis. They are able to inhibit the activation of IL-18 which in turn would decrease IL-18-mediated induction of IFN-γ. The immunostimulatory effects of IFN-γ on cell-mediated immunity are thereby inhibited. Accordingly, in one embodiment, the vaccinia virus has a mutation in a SPI including, but not limited to, B13R, B22R, and other polypeptides with similar activities or properties.

In one embodiment, one or more IL-1β-modulating polypeptides are altered in the oncolytic vaccinia virus. IL-1β is a biologically active factor that acts locally and also systemically. Blockade of the synthesis of IL-1β by the virus is regarded as a strategy allowing systemic antiviral reactions elicited by IL-1 to be suppressed or diminished. Binding proteins that effectively block the functions of IL-1 include B15R. Vaccinia virus also encodes another protein, designated B8R, which behaves like a receptor for cytokines. Accordingly, in one embodiment, the vaccinia virus has a mutation in an IL-1 modulating polypeptide including, but not limited to, B13R, B15R, and other polypeptides with similar activities or properties.

In one embodiment, the vaccinia virus administered to the subject is enriched for the EEV form of the virus. EEV has developed several mechanisms to inhibit its neutralization within the bloodstream. First, EEV is relatively resistant to complement due to the incorporation of host cell inhibitors of complement into its outer membrane coat plus secretion of vaccinia virus complement control protein (VCP) into local extracellular environment. Second, EEV is relatively resistant to neutralizing antibody effects compared to IMV. EEV is also released at earlier time points following infection (e.g., 4-6 hours) than is IMV (which is only released during/after cell death). Therefore, spread of the EEV form is faster.

Polypeptides involved in the modulation of the EEV form of a virus include, but are not limited to, A34R, B5R, and various other proteins that influence the production of the EEV form of the poxviruses. A mutation at codon 151 of the A34R gene from a lysine to an aspartic acid (K151D mutation) renders the A34R protein less able to tether the EEV form to the cell membrane. Other mutations, such as K151Q or K151E have a similar effect. B5R is an EEV-membrane bound polypeptide that may bind complement. The total deletion of A43R may lead to increased EEV release, but markedly reduced infectivity of the viruses, while the K151 mutation increases EEV release while maintaining infectivity of the released viruses.

### Adenovirus

In one embodiment, the recombinant viruses of the present invention may be an adenovirus, including all serotypes and subtypes and both naturally occurring and recombinant forms. Adenovirus has been usually employed as a gene delivery system because of its mid-sized genome, ease of manipulation, high titer, wide target-cell range, and high infectivity. Both ends of the viral genome contains 100-200 by ITRs (inverted terminal repeats), which are cis elements necessary for viral DNA replication and packaging. The E1 region (E1A and E1B) encodes proteins responsible for the regulation of transcription of the viral genome and a few cellular genes. The expression of the E2 region (E2A and E2B) results in the synthesis of the proteins for viral DNA replication.

Suitably, such adenoviruses are ones that infect human cells. Such adenoviruses may be wild-type or may be modified in various ways known in the art. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. Such modifications also include deletions of all of the coding regions of the adenoviral genome. The terms also include replication-conditional adenoviruses; that is, viruses that preferentially replicate in certain types of cells or tissues but to a lesser degree or not at all in other types. In a suitable embodiment, the adenoviral particles replicate in abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700.

Methods have been used to construct oncolytic adenoviruses: the selection of viral functions that are not necessary in tumor cells and the replacement of viral promoters with tumor-selective promoters. In one embodiment, the mutant gene is in the E1 region, and in particular, affects E1a because it controls the expression of other viral genes. For example, mutated adenovirus in E1b-55K has been used to treat tumors defective in p53 although with little clinical success owing to its low propagation capacity or oncolytic potency. In one embodiment, the mutation affects E1a. E1a mediates the bonding to proteins of the Retinoblastoma (Rb) family. pRb proteins block the transition of the Go/G1 phase to the S phase of the cell cycle, forming a complex transcription inhibitor along with E2F. When E1a bonds with a pRb, the E2F transcription factor of the pRb-E2F complex is released and E2F acts as a transcriptional activator of the genes responsible for moving on to the S phase and viral genes such as E2. The release of E2F is thus a key step in the replication of the adenovirus. In tumor cells, the cell cycle is out of control because pRb is absent or inactivated by hyperphosphorylation and E2F is free. In these cells, the inactivation of pRb by E1a is now not necessary. Thus, an adenovirus with a mutation in E1a called Delta-24 that prevents its bonding with pRb can be propagated normally in cells with inactive pRb.

A small portion of adenoviral genome is known to be necessary as cis elements (Tooza, J. Molecular biology of DNA Tumor viruses, 2nd ed. Cold Spring Harbor Laboratory, Cold Spring Harbor, N.Y. (1981)), allowing substitution of large pieces of adenoviral DNA with foreign sequences, particularly together with the use of suitable cell lines such as 293. In this context, the recombinant adenovirus comprises the adenoviral ITR sequence as an essential sequence as well as the transcription regulatory sequence for the PD-1 binding agent gene.

In a suitable embodiment, the transcription regulatory sequence for the PD-1 binding agent gene is inserted into either the deleted E1 region (E1A region and/or E1B region) or the deleted E3 region. Another foreign sequence (e.g., cytokine genes, immuno-costimulatory factor genes, apoptotic genes and tumor suppressor genes) may be additionally inserted into the recombinant adenovirus, into either the deleted E1 region (E1A region and/or E1B region) or the deleted E3 region. Furthermore, the inserted sequences may be incorporated into the deleted E4 region. In nature, adenovirus can package approximately 105% of the wild-type genome, providing capacity for about extra 2 kb of DNA. In this regard, the foreign sequences described above inserted into adenovirus may be further inserted into adenoviral wild-type genome.

In one embodiment, the recombinant adenovirus of this invention comprises the inactivated E1B 19 gene, inactivated E1B 55 gene or inactivated E1B 19/E1B 55 gene. The term “inactivation” in conjunction with genes used herein refers to conditions to render transcription and/or translation of genes to occur non-functionally, thereby the correct function of proteins encoded genes cannot be elicited. For example, the inactivated E1B 19 gene is a gene incapable of producing the functional E1B 19 kDa protein by mutation (substitution, addition, and partial and whole deletion). The defect E1B 19 gives rise to the increase in apoptotic incidence and the defect E1B 55 makes a recombinant adenovirus tumor-specific.

In one embodiment, where the recombinant adenovirus comprises the transcription activation domain, it is preferred that the recombinant adenovirus comprises an inactive DA gene; in the case that the recombinant adenovirus comprises the transcription activation domain, it is preferred that the recombinant adenovirus comprises an active DA gene. The recombinant adenovirus carrying the active DA gene is replication competent. According to another embodiment, the recombinant adenovirus comprises the inactive E1B 19 gene and active DA gene. Still more preferably, the recombinant adenovirus of this invention comprises the inactive E1B 19 gene and active DA gene, and the transcription regulatory sequence for the PD-1 binding agent gene in a deleted E3 region.

In an exemplary embodiment, the recombinant adenovirus of this invention comprises the inactive E1B gene and mutated active E1A gene, and the transcription regulatory sequence for the PD-1 binding agent gene in a deleted E3 region. It has been already suggested that tumor cells have mutated Rb and impaired Rb-related signal pathway as well as mutated p53 protein. Hence, the replication of adenoviruses lacking Rb binding capacity is suppressed in normal cells by virtue of Rb activity, whereas adenoviruses lacking Rb binding capacity actively replicate in tumor cells with repressed Rb activity to selectively kill tumor cells. In this context, the recombinant adenoviruses with the mutated Rb binding region show significant tumor specific oncolytic activity.

In another embodiment, the recombinant adenoviruses comprise a tumor specific promoter operatively linked to the active E1A gene to elevate cancer cell selectivity of E1A gene expression, permitting viruses to be propagated in more tumor-specific manner. Where the tumor specific promoter is used, TERT promoter or E2F promoter is preferable.

### Methods of Treatment and Dosage

In one aspect, the present technology provides a method for treating cancer in a subject having or at risk for having cancer. Any suitable diagnostic test and/or criteria can be used to identify the subject. For example, a subject may be considered “at risk” of a tumor if (i) the subject has a mutation, genetic polymorphism, gene or protein expression profile, and/or presence of particular substances in the blood, associated with increased risk of developing or having cancer relative to other members of the general population not having mutation or genetic polymorphism; (ii) the subject has one or more risk factors such as having a family history of cancer, having been exposed to a carcinogen or tumor-promoting agent or condition, e.g., asbestos, tobacco smoke, aflatoxin, radiation, chronic infection/inflammation, family history, etc., advanced age; (iii) the subject has one or more symptoms of cancer, etc.

Moreover, as used herein “treatment” or “treating” includes amelioration, cure, and/or maintenance of a cure (i.e., the prevention or delay of relapse) of a disorder (e.g., a tumor). Treatment after a disorder aims to reduce, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). A suitable dose and therapeutic regimen may vary depending upon the specific oncolytic virus used, the mode of delivery of the oncolytic virus, and whether it is used alone or in combination with one or more other oncolytic viruses or compounds.

In some embodiments, the cancer is a colon carcinoma, a pancreatic cancer, a breast cancer, an ovarian cancer, a prostate cancer, a squamous cell carcinoma, a cervical cancer, a lung carcinoma, a small cell lung carcinoma, a bladder carcinoma, a basal cell carcinoma, an adenocarcinoma, a sweat gland carcinoma, a sebaceous gland carcinoma, a papillary carcinoma, a papillary adenocarcinoma, a cystadenocarcinoma, a medullary carcinoma, a bronchogenic carcinoma, a renal cell carcinoma, a hepatocellular carcinoma, a bile duct carcinoma, a choriocarcinoma, a seminoma, an embryonal carcinoma, a Wilms' tumor, melanoma, a brain tumor, or a testicular tumor. Other cancers will be known to one of ordinary skill in the art.

The technology also provides methods of inducing an immune response to cancer in a patient, which involve administering to the patient a virus that expresses a PD-1 binding agent. The oncolytic virus can be administered, for example, to a tumor of the patient either by direct injection to the tumor or by system delivery. In addition, the patient can have or be at risk of developing metastatic cancer, and the treatment can be carried out to treat or prevent such cancer.

An effective amount of the oncolytic virus composition is defined herein as that amount sufficient to induce oncolysis, the disruption or lysis of a cancer cell, as well as slowing, inhibition or reduction in the growth or size of a tumor and includes the eradication of the tumor in certain instances. An effective amount can also encompass an amount that results in systemic dissemination of the therapeutic virus to tumors indirectly, e.g., infection of non-injected tumors.

To induce oncolysis, one would contact a tumor with the oncolytic virus. The routes of administration will vary, naturally, with the location and nature of the lesion, and include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration.

Intratumoral injection, or injection directly into the tumor vasculature is specifically contemplated for discrete, solid, accessible tumors. Local, regional or systemic administration also may be appropriate. For example, for tumors of >4 cm, the volume to be administered may be about 4-10 ml (suitably 10 ml), while for tumors of <4 cm, a volume of about 1-3 ml may be used (suitably 3 ml). In some embodiments, the volume of agent administered can be up to 25% or up to 33% of the tumor volume. Multiple injections delivered as single dose comprise about 0.1 to about 0.5 ml volumes. The viral particles may advantageously be contacted by administering multiple injections to the tumor, spaced at approximately 1 cm intervals. In the case of surgical intervention, the present compositions may be used preoperatively, to render an inoperable tumor subject to resection. Continuous administration also may be applied where appropriate, for example, by implanting a catheter into a tumor or into tumor vasculature. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions, particularly in the treatment of melanomas and sarcomas.

Treatment regimens may vary as well, and often depend on tumor type, tumor location, disease progression, health and age of the patient. Certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be respectable. Treatments with therapeutic viral constructs may increase the respectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 10 to 500, 100-1000, or up to about or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ or higher infectious viral particles (vp), including all values and ranges there between, to the tumor or tumor site.

### Pharmaceutical Formulations

The oncolytic virus may be prepared in a suitable pharmaceutically acceptable carrier or excipient. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### Combination Therapy

In some embodiments, it may be desirable to combine an oncolytic virus carrying a gene encoding a PD-1 binding agent with other agents effective in the treatment of cancer. For example, the treatment of a cancer may be implemented with an oncolytic virus and other anti-cancer therapies, such as anti-cancer agents or surgery. In the context of the present technology, it is contemplated that oncolytic virus therapy could be used in conjunction with chemotherapeutic, radiotherapeutic, immunotherapeutic or other biological intervention.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In some embodiments, the oncolytic virus carrying a gene encoding a PD-1 binding agent is combined with an adjuvant. In one embodiment, the adjuvant is an oligonucleotide comprising an unmethylated CpG motif. Unmethylated dinucleotide CpG motifs in bacterial deoxyribonucleic acid (DNA) have advantages for stimulating several immune cells to secrete cytokines for enhancements of innate and adaptive immunity.

The viral therapy may precede or follow the other agent treatment by intervals ranging from minutes to weeks. In embodiments where the other agent and oncolytic virus are applied separately to the cell, one would generally ensure that a significant period of time did not expire between the time of each delivery, such that the agent and virus would still be able to exert an advantageously combined effect on the cell. In such instances, it is contemplated that one may contact the cell with both modalities within about 12-24 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

### Measuring Efficacy of Oncolytic Viruses

In one aspect, the technology provides methods for determining the efficacy of an oncolytic virus for killing neoplastic cells and inducing a systemic immune response. There are many instances where it might be desirable to determine the efficacy of an oncolytic virus. For example, it may be desirable to evaluate efficacy during the development of a new oncolytic virus. It may also be desirable to evaluate efficacy of a previously developed oncolytic virus to, for example, evaluate additional properties such as shelf life, production methods, etc.

In some embodiments, the methods involve measuring the efficacy of the oncolytic in vivo. For example, the tumor may be examined using classical imaging techniques (e.g., CT and PET) before and after treatment to determine the effects of the oncolytic virus.

In some embodiments, the methods involve contacting a cancer cell with the oncolytic virus and determining the viability of the cancer cell. Cell viability may be evaluated by any one of a number of methods known in the art. For example, the viability may be evaluated in a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. It is understood that assays of cell viability are capable of detecting cell killing (i.e., cell death). Cell death may be, for example, cytolytic, apoptotic, or necrotic.

Other exemplary assays of cell viability include BrdU, EdU, or H3-Thymidine incorporation assays; DNA content assays using a nucleic acid dye, such as Hoechst Dye, DAPI, Actinomycin D, 7-aminoactinomycin D or Propidium Iodide; Cellular metabolism assays such as AlamarBlue, MTT, XTT, and CellTitre Glo; Nuclear Fragmentation Assays; Cytoplasmic Histone Associated DNA Fragmentation Assay; PARP Cleavage Assay; TUNEL staining; and Annexin staining. Still other assays will be apparent to one of ordinary skill in the art.

The cancer cells used in the efficacy evaluation methods may be any of the cancer cell lines disclosed herein and/or known in the art. In certain cases, it is desirable that the cancer cell is of a specific type. For example, it is particularly desirable that the cancer cell is a pancreatic cell when the condition to be treated by the oncolytic virus under evaluation is a pancreatic cancer.

In some cases, evaluation methods involve determining the expression of a cancer cell marker (e.g., at least one) in the cancer cell. Any appropriate cancer cell biomarker may be used. The cancer cell biomarkers can be evaluated by any appropriate method known in the art. For example, immunoblotting, immunohistochemistry, immunocytochemistry, ELISA, radioimmunoassays, proteomics methods, such as mass spectroscopy or antibody arrays may be used. In some embodiments, high-content imaging or Fluorescence-activated cell sorting (FACS) of cells may be used. Other exemplary methods will be apparent to the skilled artisan.

In some embodiments, the methods involve determining the replication of the oncolytic virus in the cancer cell. In some embodiments, the methods involve determining a spread of the oncolytic herpes virus from the cancer cell to a second cancer cell. In some cases the replication of the oncolytic herpes virus and/or the spread of the oncolytic herpes virus are determined by detecting the expression of a gene of the oncolytic virus. Any appropriate gene can be detected (e.g., endogenous, exogenous, a transgene, a reporter gene, etc.). The reporter gene may be, without limitation, a fluorescent or luminescent protein, enzyme, or other protein amenable to convenient detection and, optionally, quantitation. Examples include GFP, RFP, BFP, YFP, CYP, SFP, reef coral fluorescent protein, mFruits such as mCherry, luciferase, aequorin and derivatives of any of the foregoing. Enzyme reporter proteins such as beta-galactosidase, alkaline phosphatase, chloramphenicol acetyltransferase, etc., are also of use.

Typically, the methods for determining the efficacy of an oncolytic herpes virus for killing cancer cells are carried out in vitro under standard cell culture conditions. However, the methods are not so limited. The methods may involve growing cancer cells and optionally control cells, which may or may not be cancer cells. The cells may be grown in single well or multi-well format (e.g., 6, 12, 24, 96, 384, or 1536 well format). Thus, in some cases the assays may be adapted to a high-throughput format.

### EXAMPLES

The present compositions and methods, thus generally described, will be understood more readily by reference to the following examples, which are provided by way of illustration and are not intended to be limiting.

### Example 1

### Anti-Tumor Effects of Anti-PD-1 and Vaccinia Virus (TK−) Combination Therapy

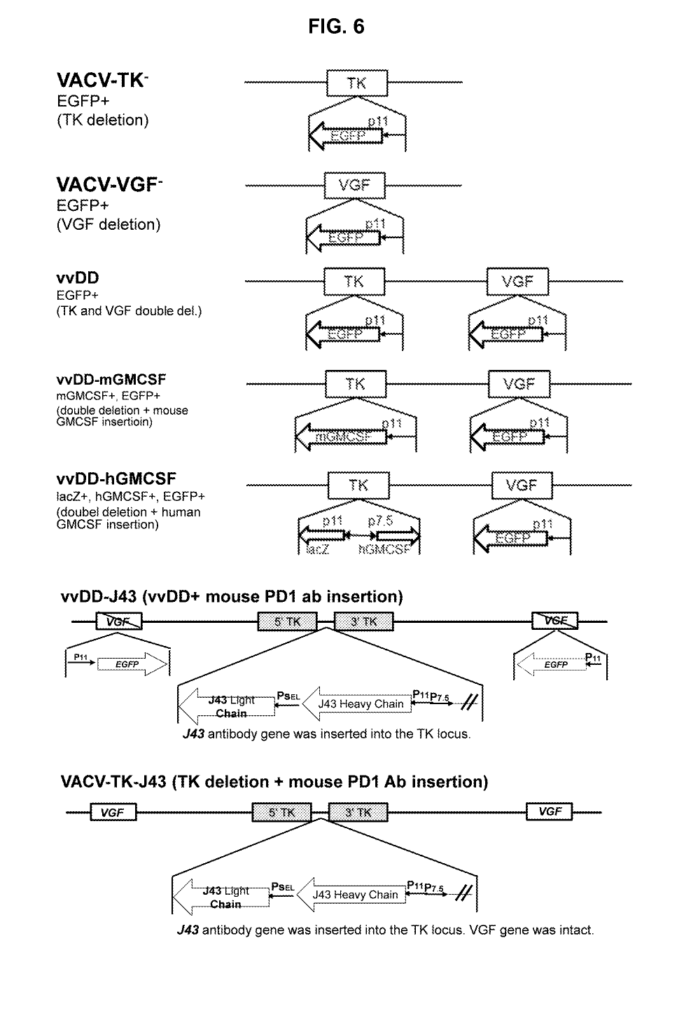

The effects of a combination of oncolytic viruses and an anti-PD-1 monoclonal antibody were assessed in vivo as follows. B16F10 melanoma cells were implanted subcutaneously in the flanks of C57BL/6 mice (3×10⁶ cells/mouse). Groups were treated with PBS, vaccinia virus TK⁻ strain (VACV-TK⁻, 10⁶ pfu/50 μl intratumorally at 0, 8, 15, and 22 days), an anti-PD-1 mouse monoclonal antibody (J43, 100 μg/200 μl IP injection every 2 days), or a combination of VACV-TK⁻ and J43 treatment. A schematic of the virus is shown in FIG. 6.

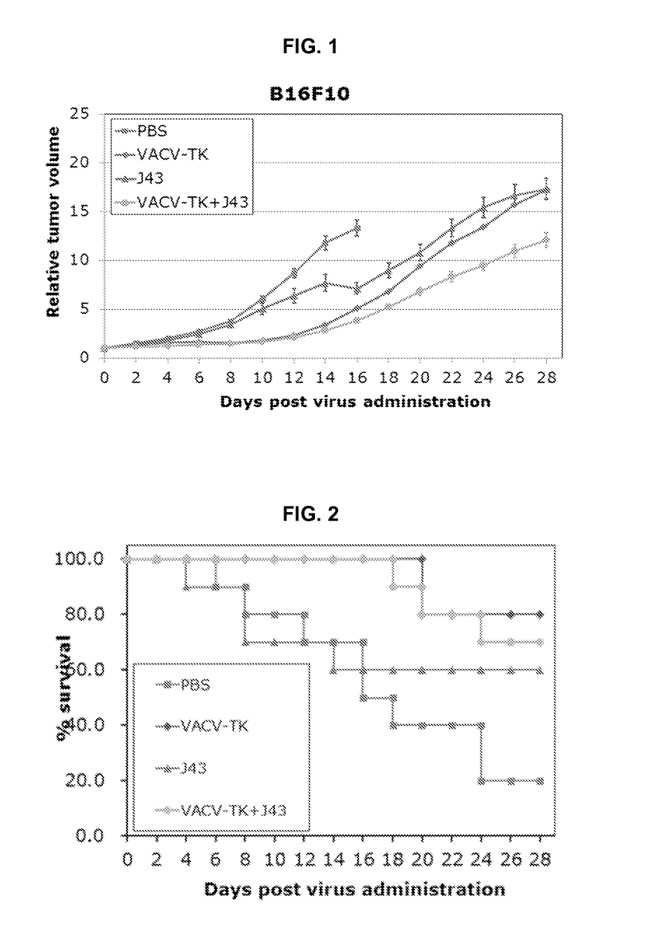

The relative tumor volume (Mean±SE) over time (n=10 per group) is shown in FIG. 1 and the survival rate of melanoma mice treated with VACV-TK⁻, J43, or VACV-TK⁻ +J43 is shown in FIG. 2. Results indicate that an improved antitumor effect can be seen from the co-injection of VACV-TK⁻ (IT) with J43 (IP). Local tumor growth of mice receiving the oncolytic virus plus antibody was significantly less than control mice or mice receiving each agent singly.

### Example 2

### Anti-Tumor Effects of Anti-PD-1 and Vaccinia Virus (vvDD) Combination Therapy

The effects of a combination therapy of oncolytic viruses and an anti-PD-1 monoclonal antibody were assessed in vivo as follows. B16F10 melanoma cells were implanted subcutaneously in the flanks of C57BL/6 mice (3×10⁶ cells/mouse). Groups were treated with PBS, vaccinia virus TK and VGF double deletion strain (vvDD, 2×10⁸ pfu/50 μl intratumorally at 0, 8, 15, and 22 days), an anti-PD-1 monoclonal antibody (J43, 100 μg/200 μl IP injection every 2 days), or a combination of vvDD and J43. A schematic of the viral strain is shown in FIG. 6.

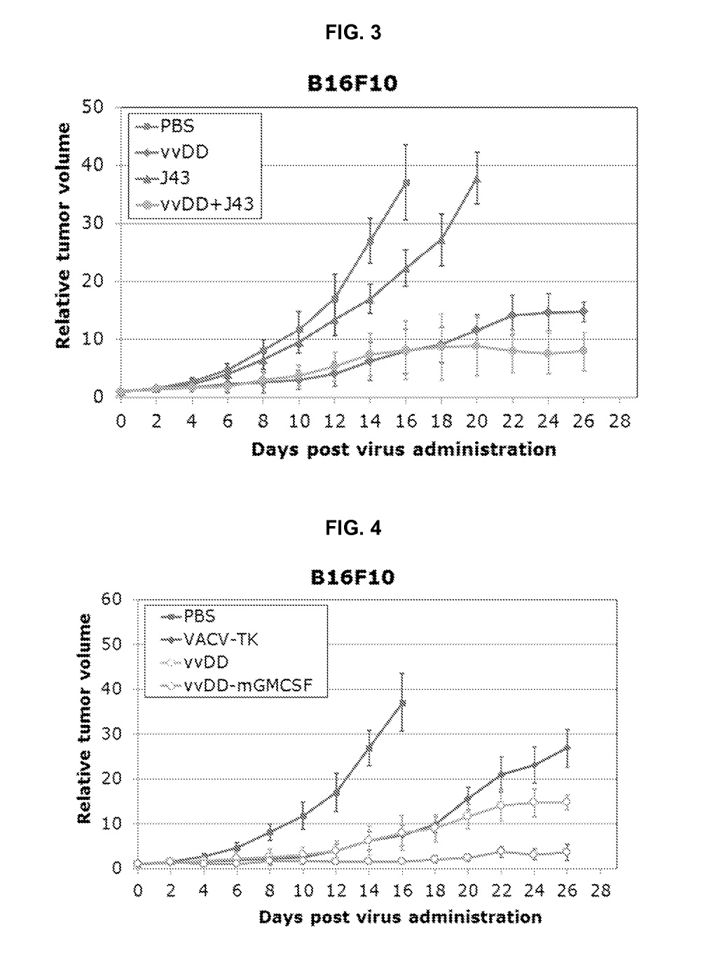

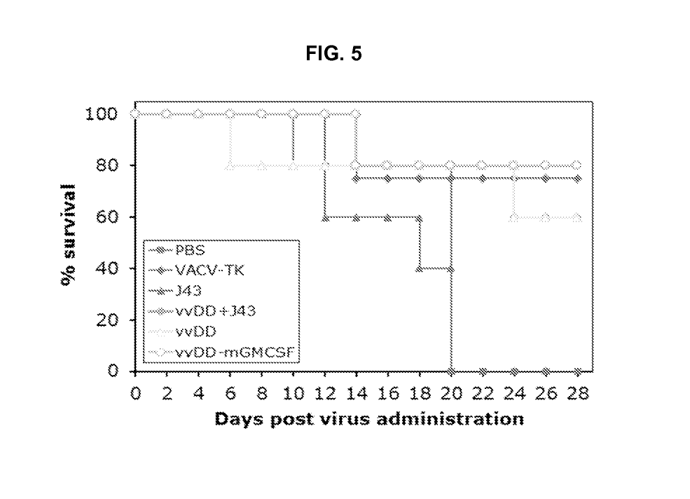

The relative tumor volume (Mean±SE) over time (n=5 per group) is shown in FIGS. 3 and 4, and the survival rate of melanoma mice treated with VACV-TK⁻, vvDD, J43, vvDD-mGMCSF, or vvDD +J43 is shown in FIG. 5. Results indicate that an improved antitumor effect can be seen from the co-injection of vvDD (IT) with J43 (IP). Local tumor growth of mice receiving the oncolytic virus plus antibody was significantly less than control mice or mice receiving each agent singly. The results also show that localized expression of an immune modulator (GMCSF) by the virus can lead to a greater benefit (FIG. 4). Consequently, these results suggest that localized expression of an anti-PD-1 monoclonal antibody would similarly lead to an enhanced effect due to increased concentration of the antibody in the tumor microenvironment.

### Example 3

### Construction of Oncolytic Vaccinia Virus Expressing PD-1 Binding Agent

In this example, oncolytic vaccinia viruses expressing a PD-1 binding agent were constructed. The oncolytic VACV-TK⁻J43 or vvDD-J43 viruses were constructed by inserting the J43 antibody gene cassette into the TK locus as shown in FIG. 6. The ligation mixture was directly transfected into HEK293 cells and incubated to permit the generation of infectious virus. The resultant viruses are subsequently plaque-purified.

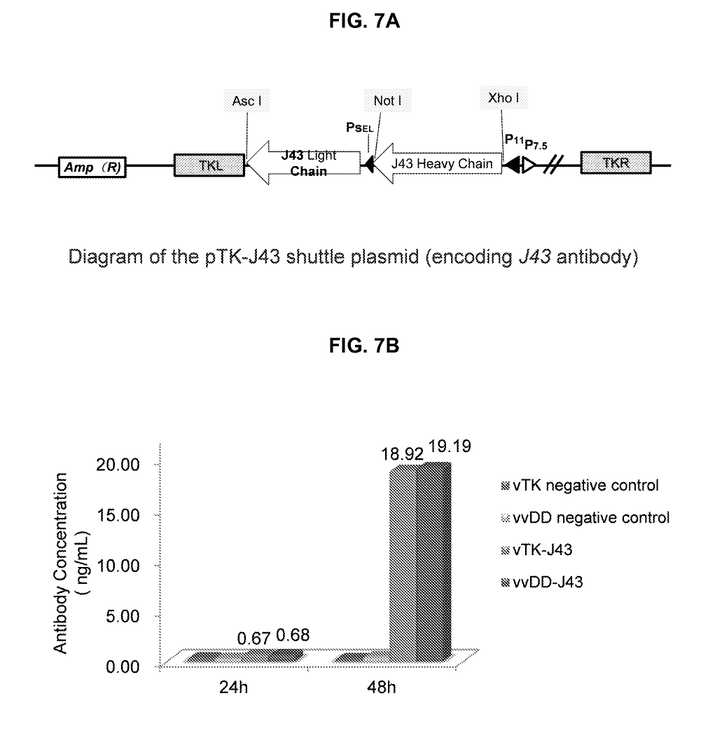

In order to check whether or not the constructed mPD1 mAb, J43, can be expressed, HEK293 cells were transiently transfected by plasmid DNA pTK-J43 (FIG. 7A), and infected with VACV-TK− or vvDD. At 48 hr of post-infection, J43 antibody expression can be detected in cell culture medium by ELISA, indicating secreted J43 antibody was released into the medium (FIG. 7B). The expression level of J43 was 19.19 ng/ml in vvDD-J43, and 18.92 ng/ml in VACV-TK⁻-J43.

### Example 4

### Construction of Oncolytic HSV-1 Expressing PD-1 Binding Agent (HSV1-PD-1)

In this example, an oncolytic HSV-1 virus expressing a PD-1 binding agent is constructed. The oncolytic HSV1-PD-1 is derived using a BAC-based construct that contains a mutated HSV genome, in which the diploid gene encoding γ34.5 and both copies of HSV packaging signal have been deleted. Infectious HSV cannot be generated from this construct unless an intact HSV packaging signal is provided in cis; otherwise, the virus will be replication conditional due to the deletion of both copies of γ34.5.

HSV1-PD-1 is constructed by inserting a DNA sequence containing a HSV packaging signal and a PD-1 binding agent gene cassette into a restriction site located in the BAC sequence. The PD-1 binding agent gene cassette encodes a PD-1 scFv fragment (prepared as described above), which is under control of either the CMV promoter or a late stage viral promoter. The ligation mixture is directly transfected into Vero cells and incubated for 3-5 days to permit the generation of infectious virus. The resultant viruses are subsequently plaque-purified. Viral stocks are prepared by infecting Vero cells with 0.01 pfu/cell. The medium is then collected and subjected to a low-speed centrifugation at 1,000×g for 10 min. The cleared supernatant is transferred to another tube, and the virus is pelleted through high-speed centrifugation (29,000×g for 4 h). The viral pellet is resuspended in PBS containing 10% glycerol and stored at −80° C.

Exemplary HSV1-PD-1 genotypes are shown in Table 2 below.

TABLE 2
Exemplary HSV-1 Constructs

| Exemplary Strain | Genotype |
|---|---|
| HSV1-PD-1 Construct #1 | HSV1 ΔICP34.5 PD-1-scFv+ |
| HSV1-PD-1 Construct #2 | HSV1 ΔICP34.5 ΔICP47 PD-1-scFv+ |
| HSV1-PD-1 Construct #3 | HSV1 ΔICP34.5 GM-CSF+ PD-1-scFv+ |
| HSV1-PD-1 Construct #4 | HSV1 ΔICP34.5 ΔICP47 GM-CSF+ |
| | PD-1-scFv+ |

### Example 5

### Construction Oncolytic HSV-2 Expressing PD-1 Binding Agent (HSV2-PD-1)

In this example, an oncolytic HSV-2 virus expressing a PD-1 binding agent protein is constructed. To construct oncolytic HSV2-PD-1, the ICP10 left-flanking region of the wild-type (wt) HSV-2 genome (equivalent to nucleotide span 85994-86999 in the HSV-2 genome), the ribonucleotide reductase domain and the right-flanking region (equivalent to nucleotide span 88228-89347) are amplified by PCR. These PCR products are cloned together to form a new plasmid containing a mutated ICP10 gene, in which the protein kinase domain (equivalent to nucleotide span 86999-88228) is deleted. A PD-1 binding agent gene cassette encoding the PD-1 scFv polypeptide, which is under control of either the CMV promoter or a late stage viral promoter, is added to the construct. The construct is inserted into the genome of wild-type HSV-2 by homologous recombination. The recombinant virus is identified by screening plaques. Viral stocks are prepared by infecting Vero cells with 0.01 plaque-forming units (pfu) per cell, harvesting the virus after 2 days, and storing it at −80° C.

Exemplary HSV-2 PD-1 genotypes are shown in Table 3 below.

TABLE 3
Exemplary HSV-2 Constructs

| Exemplary Strain | Genotype |
|---|---|
| HSV2-PD-1 Construct #1 | HSV2 ΔPK-ICP10 PD-1-scFv+ |
| HSV2-PD-1 Construct #2 | HSV2 ΔPK-ICP10 ΔICP34.5 PD-1-scFv+ |
| HSV2-PD-1 Construct #3 | HSV2 ΔPK-ICP10 ΔICP34.5 ΔICP47 |
| | PD-1-scFv+ |
| HSV2-PD-1 Construct #4 | HSV2 ΔPK-ICP10 ΔICP47 PD-1-scFv+ |
| HSV2-PD-1 Construct #5 | HSV2 ΔPK-ICP10 GM-CSF+ PD-1-scFv+ |
| HSV2-PD-1 Construct #6 | HSV2 ΔPK-ICP10 ΔICP34.5 GM-CSF+ |
| | PD-1-scFv+ |
| HSV2-PD-1 Construct #7 | HSV2 ΔPK-ICP10 ΔICP34.5 ΔICP47 |
| | GM-CSF+ PD-1-scFv+ |
| HSV2-PD-1 Construct #8 | HSV2 ΔPK-ICP10 ΔICP47 GM-CSF+ |
| | PD-1-scFv+ |

### Example 6

### Cell Culture Assays

Human cancer cell lines representing each of the major histopathological types, are obtained from American Type Culture Collection (Rockville, Md.) and maintained as recommended. Exemplary cell lines include any of the NCI-60 cell lines or any of the cell lines set forth in Table 5 below.

TABLE 5
Exemplary Cancer Cell Lines

| Name | Description |
|---|---|
| H460 | human large cell lung cancer |
| Hep 3B | human HCC cell line |
| Hep G2 | human HCC cell line |
| Skov3 | human ovarian cancer cell line |
| PA1 | human ovarian teratocarcinoma cell line |
| MDA435 | human breast cancer cell lines |
| U20S | human osteosarcoma cell line lacking p16 |
| HCT 116 | Colon Cancer Cell Line |
| U87 | human glioma cell line |
| Mpan 96 | Pancreatic carcinoma cell line |
| Hela | human cervical cancer cell line |
| MCF-7 | human breast cancer cell line |
| SK-Mel-28 | human, skin, melanoma |
| Eca-109 | human esophageal carcinoma cells |
| PC-2 | human pancreatic tumor cells |
| PC-3 | human prostate cancer cells |

The effect of each of the oncolytic viruses in the cancer cell lines is assessed as follows. Cells are incubated in 96-well plates at a density of about 3,000 cells per well. Twenty-four hours later, the cells are infected at selected values of multiplicity of infection (MOI) for one hour in serum-free medium. When the cells in the control well are confluent (i.e. generally between days 3 and 6), the percentage of viable cells is assessed in all wells.

Cell viability is assessed by colorimetric assay, using a CellTiter 96 Aqueous kit obtained from Promega (Madison, Wis.) per the manufacturer's instructions. Each treatment experiment is performed at least twice. It is predicted that the viability of cells treated with the oncolytic virus will be significantly less than control cells.

### Example 7

### In Vivo Xenograft Model Studies

The effects of the oncolytic viruses are assessed in vivo as follows. Cancer cell lines are implanted subcutaneously in the flanks of either immunocompetent or severe combined immunodeficient (SCID) mice. The tumors which develop have a mean volume of 160 to 170 cubic millimeters. Oncolytic virus is either injected directly systemically at a dose of 10⁴-10⁸ pfu or injected into individual tumors at a dose of 10⁴-10⁸ pfu. Control mice are injected with medium alone. Tumor volume is estimated in all mice at regular intervals. Tumor volume is calculated by the formula tumor volume [mm³]=(length [mm])×(width [mm])²×0.52. Tumor growth curves are generated using the estimated values for tumor volume. After a period of 3-4 weeks, mice are sacrificed and their tumors are weighed. It is predicted that local tumor growth of mice receiving the oncolytic virus will be significantly less than control mice.

### Example 8

### Metastatic Breast Cancer Model

The effects of the oncolytic viruses are assessed in the 4T1 metastatic breast cancer model as follows. This model is a syngeneic xenograft model based on 4T1-12B, a luciferase-expressing clone of the well characterized 4T1 mouse mammary tumor cell line. The luciferase-expressing line is introduced orthotopically into the mammary fat pad of nude, scid or normal BALB/c mice by surgery or direct injection, intravenously by tail vein injection, or arterially by surgical catheterization of the right carotid artery. When introduced orthotopically, the 4T1 cell grows rapidly at the primary site and forms metastases in lungs, liver, bone and brain over a period of 3-6 weeks. When introduced via the tail vein or arterially, metastases are apparent in these same organs after 1-2 weeks. Oncolytic virus is either injected directly systemically at a dose of 10⁴-10⁸ pfu or injected into individual tumors at a dose of 10⁴-10⁸ pfu. Control mice are injected with medium alone. It is predicted that metastatic tumor growth of mice receiving the oncolytic virus will be significantly less than control mice.

### Example 9

### Mouse Melanoma Model

C57BL/6 mice bearing B16-F10 melanoma tumors are administered oncolytic virus either systemically at a dose of 10⁴-10⁸ pfu or the virus is injected into individual tumors at a dose of 10⁴-10⁸ pfu. Tumor growth is monitored and antigen-specific splenocyte responses are assayed by ELISPOT. It is predicted that local tumor growth of mice receiving the oncolytic virus will be significantly less than control mice.

### Example 10

### HCC Model

Transgenic mice bearing hepatitis C virus core or the HBx protein of hepatitis B virus are used to induce HCC in a murine subject (See Heindryckx et al., Experimental mouse models for hepatocellular carcinoma research, Int. J. Exp. Path (2009) 90:367-386). Tumors are administered oncolytic virus either systemically at a dose of 10⁴-10⁸ pfu or the virus is injected into individual tumors at a dose of 10⁴-10⁸ pfu. Tumor growth is monitored and antigen-specific splenocyte responses are assayed by ELISPOT. It is predicted that local tumor growth of mice receiving the oncolytic virus will be significantly less than control mice.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 particles refers to groups having 1, 2, or 3 particles. Similarly, a group having 1-5 particles refers to groups having 1, 2, 3, 4, or 5 particles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference in their entireties and for all purposes to the same extent as if each individual publication, patent, or patent application was specifically and individually incorporated by reference in its entirety for all purposes.

1. A recombinant oncolytic virus comprising a heterologous nucleic acid sequence encoding an anti-PD-1 binding protein that antagonizes the activity of PD-1, wherein the heterologous nucleic acid sequence is stably incorporated into the genome of the oncolytic virus.

2. The recombinant oncolytic virus of claim 1, wherein the anti-PD-1 binding protein is selected from the group consisting of a natural ligand, a genetically modified ligand, a recombinant soluble domain of a natural receptor and a modified version thereof, a peptide ligand, a polypeptide ligand, an antibody molecule and fragments and derivatives thereof, and an antibody-like molecule.

3. The recombinant oncolytic virus of claim 1, wherein the anti-PD-1 binding protein is an anti-PD-1 antibody.

4. The recombinant oncolytic virus of claim 1 wherein the oncolytic virus is selected from the group consisting of vesicular stomatitis virus (VSV), Newcastle disease virus (NDV), retrovirus, reovirus, measles virus, Sinbis virus, influenza virus, herpes simplex virus (HSV), vaccinia virus, and adenovirus.

5. The recombinant oncolytic virus of claim 4, wherein the virus is HSV.

6. (canceled)

7. The recombinant oncolytic virus of claim 5, wherein the HSV genome has a mutation in each ICP34.5 locus such that the HSV cannot express a functional ICP34.5 gene product.

8. The recombinant oncolytic virus of claim 5, wherein the HSV is HSV-2 and the HSV-2 genome encodes a modified ICP10 polypeptide having ribonucleotide reductase activity, but lacking protein kinase activity.

9. The recombinant oncolytic virus of claim 8, wherein the modified ICP10 polypeptide has a deletion in the protein kinase domain of ICP10.

10. (canceled)

11. (canceled)

12. The recombinant oncolytic virus of claim 1 further comprising a gene encoding an immunomodulatory protein selected from the group consisting of: tumor necrosis factor, interferon alpha, interferon gamma, IL-2, IL-12, IL-15, IL-24, and GM-CSF.

13. The recombinant oncolytic virus of claim 4, wherein the virus is a vaccinia virus.

14. The recombinant oncolytic virus of claim 13, wherein the virus comprises an inactivating mutation in a thymidine kinase (TK) gene to produce a negative TK phenotype.

15. The recombinant oncolytic virus of claim 13, wherein the vaccinia virus does not express functional vaccinia growth factor (VGF).

16. The recombinant oncolytic virus of claim 13, wherein the vaccinia virus does not express functional B13R.

17. The recombinant oncolytic virus of claim 13 further comprising a gene encoding an immunomodulatory protein selected from the group consisting of: tumor necrosis factor, interferon alpha, interferon gamma, IL-2, IL-12, IL-15, IL-24, and GM-CSF.

18. The recombinant oncolytic virus of claim 1, wherein the heterologous nucleic acid sequence encoding the PD-1 binding agent is operably linked to a CMV promoter.

19. The recombinant oncolytic virus of claim 4, wherein the virus is an adenovirus.

20. A pharmaceutical composition comprising an effective amount of the virus of claim 1 and a pharmaceutically acceptable carrier.

21. The pharmaceutical composition of claim 20, wherein the composition is formulated for parenteral administration.

22. The pharmaceutical composition of claim 20, wherein the composition is formulated for intratumoral administration.

23. A method of treating cancer comprising administering to a subject in need thereof an effective amount of the pharmaceutical composition of claim 20.
